(19) United States
(12) Patent Application Publication
(10) Pub. No.: US 2008/0108990 A1
(43) Pub. Date: May 8, 2008

(54) INTERSPINOUS PROCESS IMPLANT HAVING A FIXED WING AND A DEPLOYABLE WING AND METHOD OF IMPLANTATION

(75) Inventors: Steven T. Mitchell, Pleasant Hill, CA (US); Scott A. Yerby, Montara, CA (US); James F. Zucherman, San Francisco, CA (US); Ken Y. Hsu, San Francisco, CA (US); Henry A. Klyce, Piedmont, CA (US); Charles J. Winslow, Walnut Creek, CA (US); John J. Flynn, West Milford, NJ (US); John A. Markwart, Castro Valley, CA (US)

Correspondence Address: COATS & BENNETT/MEDTRONICS, 1400 CRESCENT GREEN, SUITE 300, CARY, NC, 27518, US

(73) Assignee: ST. FRANCIS MEDICAL TECHNOLOGIES, INC., Alameda, CA (US)

(21) Appl. No.: 11/556,071

(22) Filed: Nov. 2, 2006

(51) Int. Cl. A61B 17/58; A61F 2/44

(52) U.S. Cl. 606/61; 606/63; 623/17.16

(57) ABSTRACT

An embodiment of a system in accordance with the present invention can include an implant having a first wing, a spacer with a thickness and a second wing, wherein a first configuration of the second wing has a first height substantially similar to the thickness and wherein the second wing is adapted to be selectably arranged in a second configuration such that the second wing has a second height greater than the first height. The implant is then urged into position between adjacent spinous processes and subsequently arranged in a second configuration to fix the implant in position.

### CROSS-REFERENCE TO RELATED APPLICATIONS

This U.S. Patent Application incorporates by reference all of the following co-pending applications and issued patents:

U.S. Provisional Patent Application No. 60/672,402 entitled “Interspinous Process Implant Having Deployable Wings and Method of Implantation,” by Zucherman et al., filed Apr. 18, 2005 (Attorney Docket No. SFMT-01096US0);

U.S. patent application Ser. No. 10/850,267 entitled “Distractible Interspinous Process Implant and Method of Implantation,” by Zucherman et al., filed May 20, 2004 (Attorney Docket No. SFMT-01087US2);

U.S. patent application Ser. No. 11/095,680 entitled “Interspinous Process Implant Including a Binder and Method of Implantation,” by Zucherman et al., filed Mar. 31, 2005 (Attorney Docket No. SFMT-01109US1);

U.S. patent application Ser. No. 11/389,002 entitled “Interspinous Process Implant Having Deployable Wings and Method of Implantation,” by Zucherman et al., filed Mar. 24, 2006 (Attorney Docket No. SFMT-01096US1);

U.S. Patent Application No. 60/853,963 entitled “System and Methods for In Situ Assembly of an Interspinous Process Distraction Implant,” by Mitchell et al., filed Oct. 24, 2006 (Attorney Docket No. SFMT-01152US0);

U.S. Pat. No. 6,419,676, entitled “Spine Distraction Implant and Method,” issued Jul. 16, 2002 to Zucherman, et al.;

U.S. Pat. No. 6,451,019, entitled “Supplemental Spine Fixation Device and Method,” issued Sep. 17, 2002 to Zucherman, et al.;

U.S. Pat. No. 6,582,433, entitled “Spine Fixation Device and Method,” issued Jun. 24, 2003 to Yun;

U.S. Pat. No. 6,652,527, entitled “Supplemental Spine Fixation Device and Method,” issued Nov. 25, 2003 to Zucherman, et al;

U.S. Pat. No. 6,695,842, entitled “Interspinous Process Distraction System and Method with Positionable Wing and Method,” issued Feb. 24, 2004 to Zucherman, et al;

U.S. Pat. No. 6,699,246, entitled “Spine Distraction Implant,” issued Mar. 2, 2004 to Zucherman, et al; and

U.S. Pat. No. 6,712,819, entitled “Mating Insertion Instruments for Spinal Implants and Methods of Use,” issued Mar. 30, 2004 to Zucherman, et al.

### TECHNICAL FIELD

This invention relates to interspinous process implants.

### BACKGROUND OF THE INVENTION

The spinal column is a bio-mechanical structure composed primarily of ligaments, muscles, vertebrae and intervertebral disks. The bio-mechanical functions of the spine include: (1) support of the body, which involves the transfer of the weight and the bending movements of the head, trunk and arms to the pelvis and legs, (2) complex physiological motion between these parts, and (3) protection of the spinal cord and the nerve roots.

As the present society ages, it is anticipated that there will be an increase in adverse spinal conditions which are characteristic of older people. By way of example only, with aging comes an increase in spinal stenosis (including, but not limited to, central canal and lateral stenosis), and facet arthropathy. Spinal stenosis results in a reduction foraminal area (i.e., the available space for the passage of nerves and blood vessels) which compresses the cervical nerve roots and causes radicular pain. Humpreys, S. C. et al., Flexion and traction effect on C5-C6 for aminal space, Arch. Phys. Med. Rehabil., vol. 79 at 1105 (September 1998). Another symptom of spinal stenosis is myelopathy, which results in neck pain and muscle weakness. Id. Extension and ipsilateral rotation of the neck further reduces the foraminal area and contributes to pain, nerve root compression and neural injury. Id.; Yoo, J. U. et al, Effect of cervical spine motion on the neuroforaminal dimensions of human cervical spine, Spine, vol. 17 at 1131 (Nov. 10, 1992). In contrast, neck flexion increases the foraminal area. Humpreys, S. C. et al., at 1105. Pain associated with stenosis can be relieved by medication and/or surgery. It is desirable to eliminate the need for major surgery for all individuals, and in particular, for the elderly.

Accordingly, a need exists to develop spine implants that alleviate pain caused by spinal stenosis and other such conditions caused by damage to, or degeneration of, the cervical spine. Such implants would distract, or increase the space between, the vertebrae to increase the foraminal area and reduce pressure on the nerves and blood vessels of the cervical spine.

A further need exists for development of a minimally invasive surgical implantation method for cervical spine implants that preserves the physiology of the spine.

Further, a need exists for an implant that accommodates the distinct anatomical structures of the spine, minimizes further trauma to the spine, and obviates the need for invasive methods of surgical implantation. Additionally, a need exists to address adverse spinal conditions that are exacerbated by spinal extension.

### BRIEF DESCRIPTION OF THE DRAWINGS

Further details of embodiments of the present invention are explained with the help of the attached drawings in which:

FIG. 1 is a perspective view of an embodiment of an implant in accordance with the present invention having a first wing and a second wing that can be deployed after arranging the implant between adjacent spinous processes.

FIG. 2A is a posterior view of the implant of FIG. 1 positioned between adjacent spinous processes in an undeployed configuration; FIG. 2B is a posterior view of the implant of FIG. 1 positioned between adjacent spinous processes in a deployed configuration.

FIG. 3 is a posterior view of the implant of FIG. 1 positioned between two cervical vertebrae by way of a cannula.

FIG. 4 is a flowchart of an embodiment of a method in accordance with the present invention for implanting an implant as shown in FIG. 1.

FIG. 5 is a perspective view of still another embodiment of an implant in accordance with the present invention having a fixed first wing and a second wing that can be deployed after arranging the implant between adjacent spinous processes.

FIG. 6A is a posterior view of the implant of FIG. 5 positioned between adjacent spinous processes in an undeployed configuration; FIG. 6B is a posterior view of the implant of FIG. 5A positioned between adjacent spinous processes in a deployed configuration; FIG. 6C is a perspective view of a still further embodiment of the implant having a second wing and a spacer positionable by way of a cannula; FIG. 6D is a perspective view of the implant of FIG. 6C having the second deployed and a first wing connected along the rod.

FIG. 7 is a posterior view of the implant of FIG. 6 positioned between two cervical vertebrae by way of a cannula.

FIG. 8A is a flowchart of an embodiment of a method in accordance with the present invention for implanting an implant as shown in FIG. 6.

FIG. 8B is a flowchart of an alternative embodiment of a method in accordance with the present invention for implanting an implant as shown in FIG. 6.

FIG. 9A is a posterior view of a still further embodiment of an implant in accordance with the present invention having a first and second wing that can be deployed after arranging the implant between adjacent spinous processes, and a spacer that can be deployed to achieve a desired height; FIG. 9B is a posterior view of the implant of FIG. 9A positioned between adjacent spinous processes in a partially deployed configuration; FIG. 9C is a posterior view of the implant of FIG. 9A positioned between adjacent spinous processes in a deployed configuration.

FIG. 10A is a perspective view of a support portion of the spacer of the implant of FIG. 9; FIG. 10B is a perspective view of a distraction element of the spacer of the implant of FIG. 9.

FIG. 11 is a posterior view of the implant of FIG. 9 positioned between two cervical vertebrae by way of a cannula.

FIG. 12 is a flowchart of an embodiment of a method in accordance with the present invention for implanting an implant as shown in FIG. 9.

### DETAILED DESCRIPTION

### Implants Having Deployable Wings

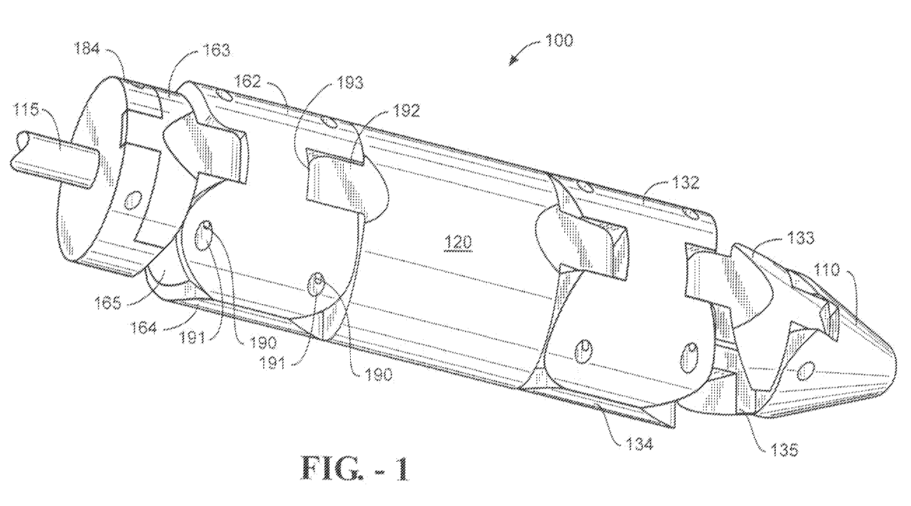

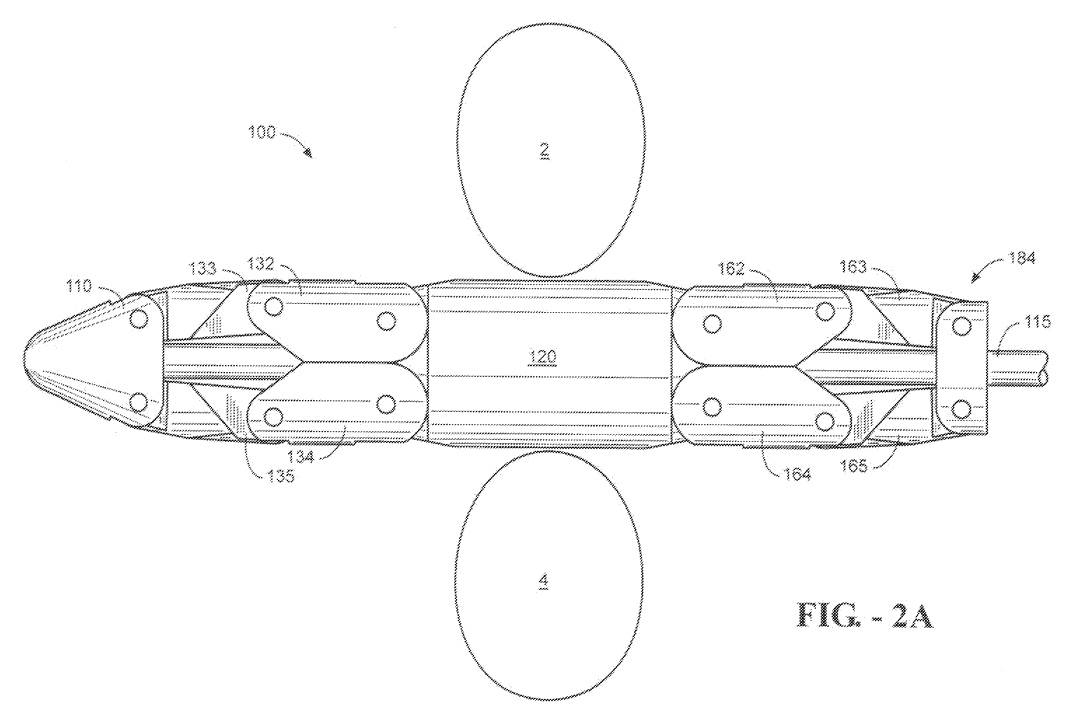

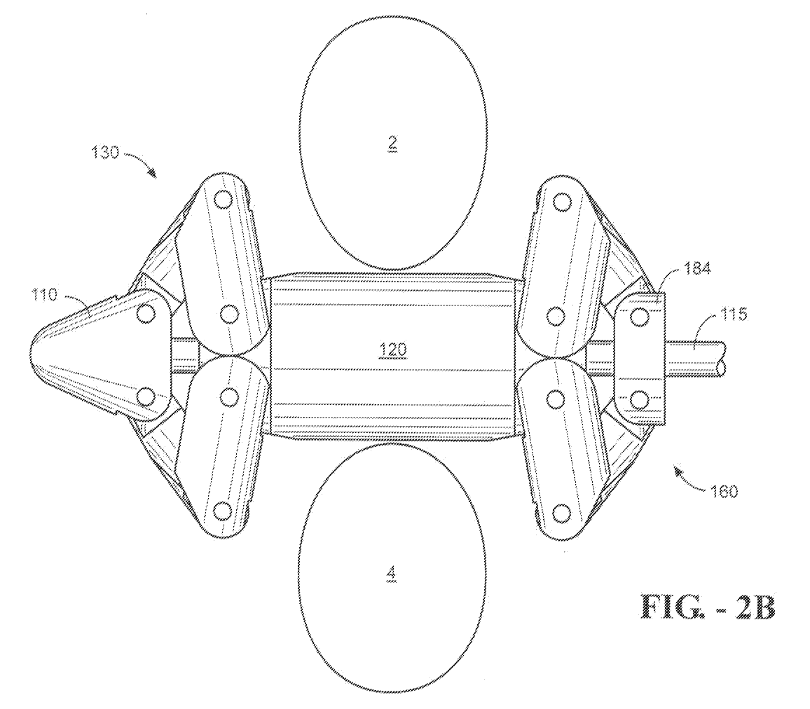

FIG. 1 is a perspective view and FIGS. 2A and 2B are posterior side views of an embodiment of an implant 100 in accordance with the present invention. The implant 100 can comprise a collapsed structure of hinged or otherwise pivotably connected segments 132-135,162-165 that when deployed (as shown in FIG. 2B) form stops 130,160 (also referred to herein as first and second wings). The first and second wings 160,130 resist undesired movement when the implant 100 is positioned between adjacent spinous processes 2,4. The implant 100 includes a spacer 120 that limits extension motion of two (or more) adjacent spinous processes 2,4 by resisting compressive forces applied to the spacer 120 by the adjacent spinous processes 2,4. The spacer 120 limits movement to preferably limit the collapse of the foraminal canal within which nerves are disposed.

In an embodiment, the segments 132-135,162-165 include complementary structures 192,193 that can be pivotably connected by pins 190 disposed within holes 191 aligned to receive the pins 190 without obstruction (i.e. they are hinged together). The spacer 120 likewise includes a complementary structure 192 for pivotably joining adjacent segments 132,134,162,164. Still further, an end piece 184 and distraction guide (also referred to herein as a tissue expander) 110 include complementary structures 192 for pivotably joining adjacent segments 163,165, 133,135.

As can be seen in FIGS. 2A and 2B, the segments 132-135,162-165 are shaped to allow a desired amount of pivoting. For example, the segments 132,134,162,164 pivotably connected with the spacer 120 have rounded shapes that curve away from the pins 190 joining the segments 132-135,162-165 so that during pivoting, the segments 132-135,162-165 have a desired range of motion without obstruction.

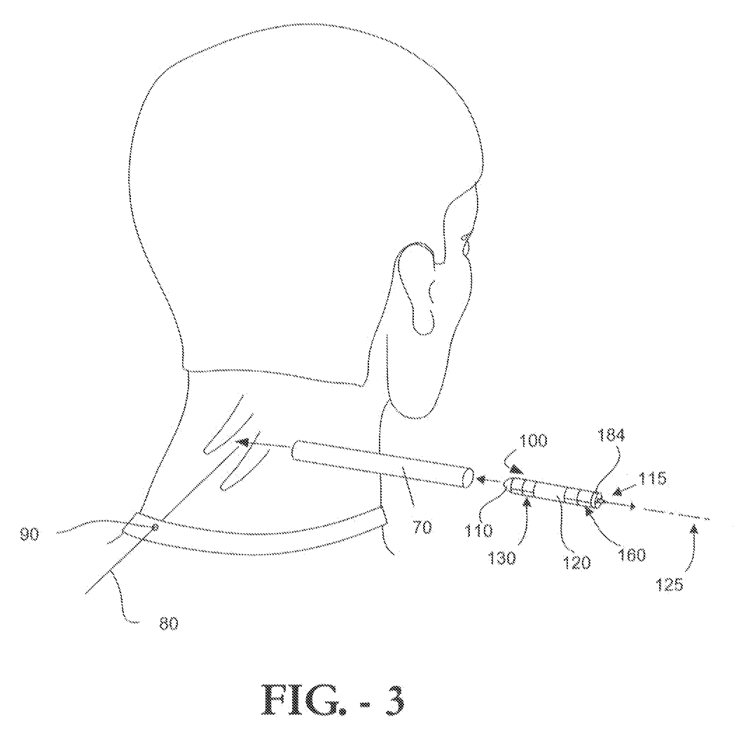

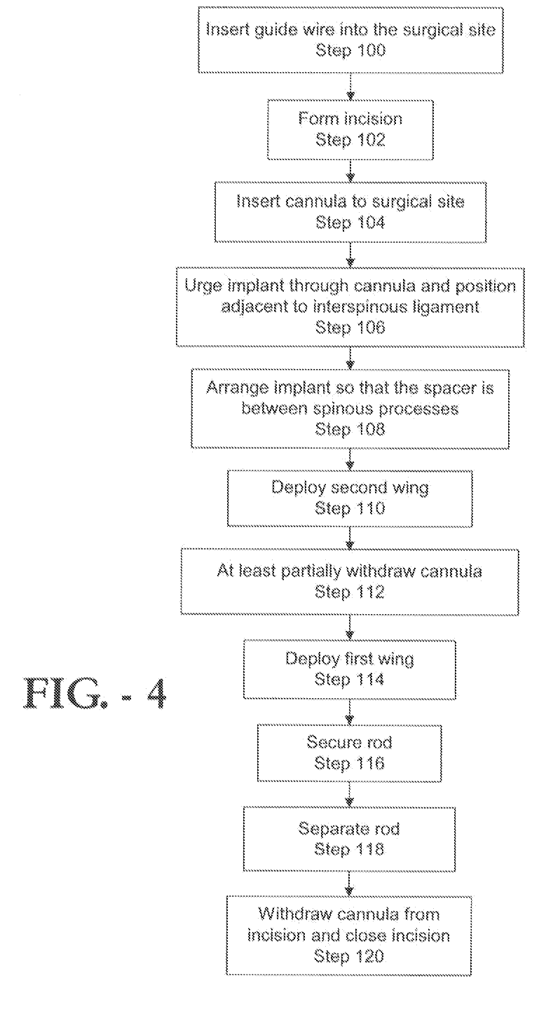

The embodiment of FIGS. 1-3 can have a first, collapsed configuration and a second, deployed configuration (as shown in FIG. 2B). Arranged in the first configuration, such implants 100 can have a substantially collapsed profile having an approximately uniform thickness. The uniform thickness approximates the thickness of the spacer 120. As shown, in the first, collapsed configuration, the implant 100 has a roughly oval cross-sectional shape approximating a cross-sectional shape of the spacer 120. Referring to FIG. 3, the first, collapsed configuration of the implant 100 allows the implant 100 to be positioned at a surgical site by way of one or more incisions made approaching the interspinous ligament from one side of the interspinous ligament. The distraction guide 110 of the implant 100 can pierce the interspinous ligament and proceed through the interspinous ligament along a longitudinal axis 125, distracting the adjacent spinous processes 2,4 of the targeted motion segment, where desired. The implant 100 can be delivered with the spacer 120 disposed between the adjacent spinous processes 2,4 without the collapsed segments 162-165 substantially obstructing movement along the longitudinal axis 125. As further shown in FIG. 3, the first, collapsed configuration can enable implantation at a surgical site by way of a cannula. An incision sized to receive the cannula can be made, and the cannula can be positioned at or near the surgical site. The cannula can have a cross-sectional shape generally conforming with a shape of the implant 100 to assist in orienting the implant 100 as desired. For example, the cannula can have an oval shape generally conforming with the oval shape of the spacer 120 of the implant 100.

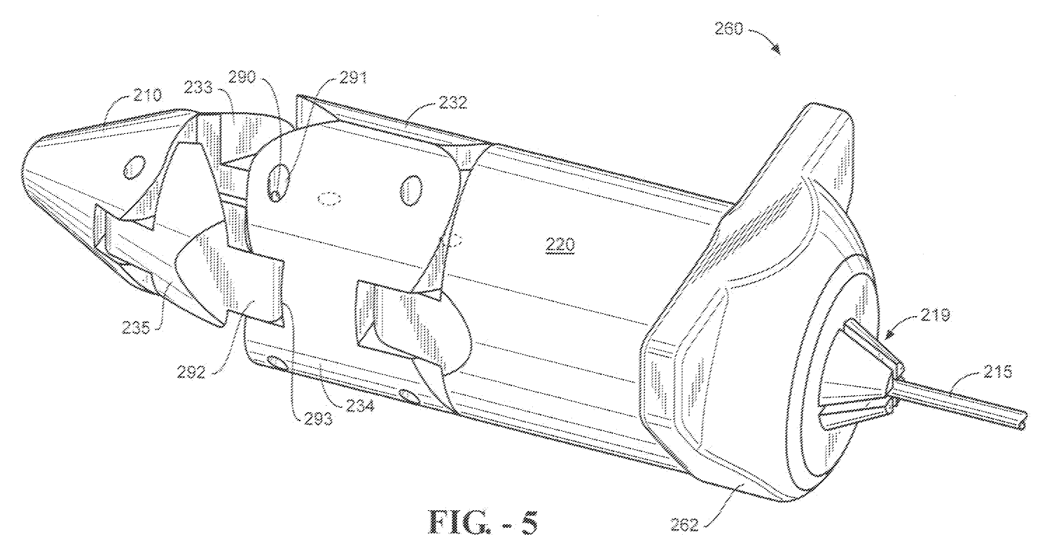

Referring to FIGS. 3 and 4, in an embodiment of a method of implantation in accordance with the present invention, the cannula can be positioned adjacent to the interspinous ligament of the targeted motion segment. Preferably a guide wire 80 is inserted through a placement network or guide 90 into the surgical site of the implant recipient (e.g. the neck where the targeted motion segment includes cervical vertebra) (Step 100). The guide wire 80 is used to locate where the implant 100 is to be placed relative to the spine, including the spinous processes. Once the guide wire 80 is positioned with the aid of imaging techniques, an incision is made (Step 102) so that the cannula 70 can be positioned through the incision and along a line that is about perpendicular to the guide wire 80 and directed at the end of the guide wire 80 (Step 104).

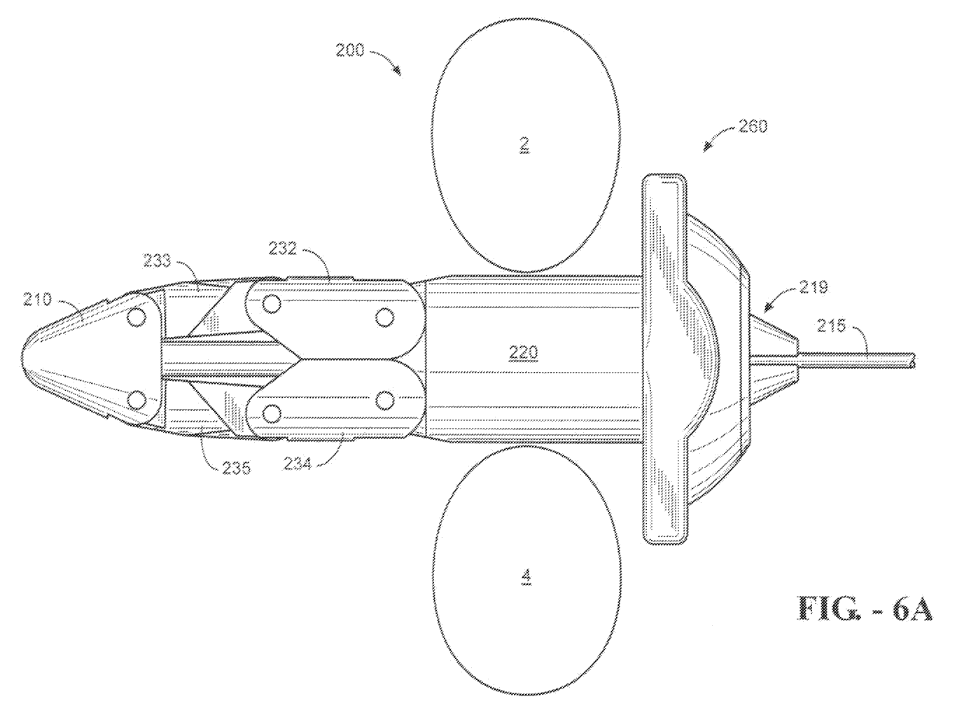

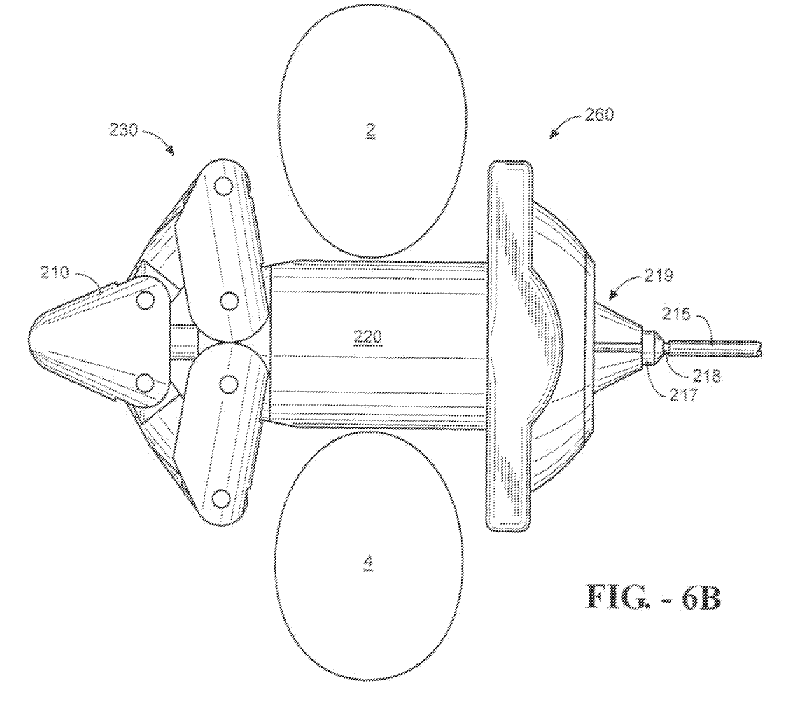

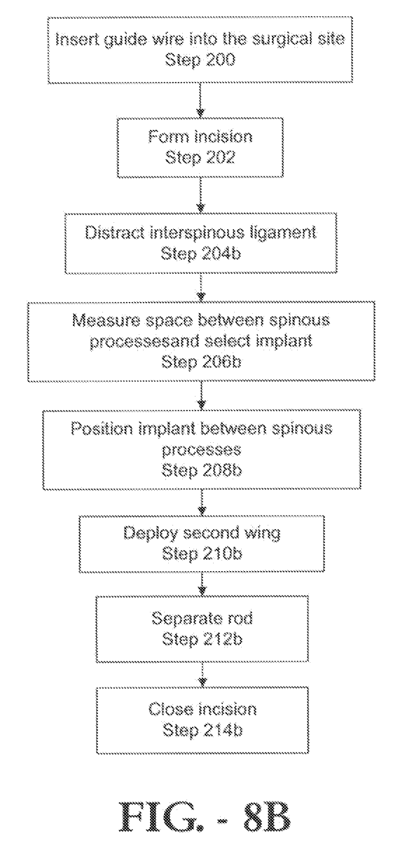

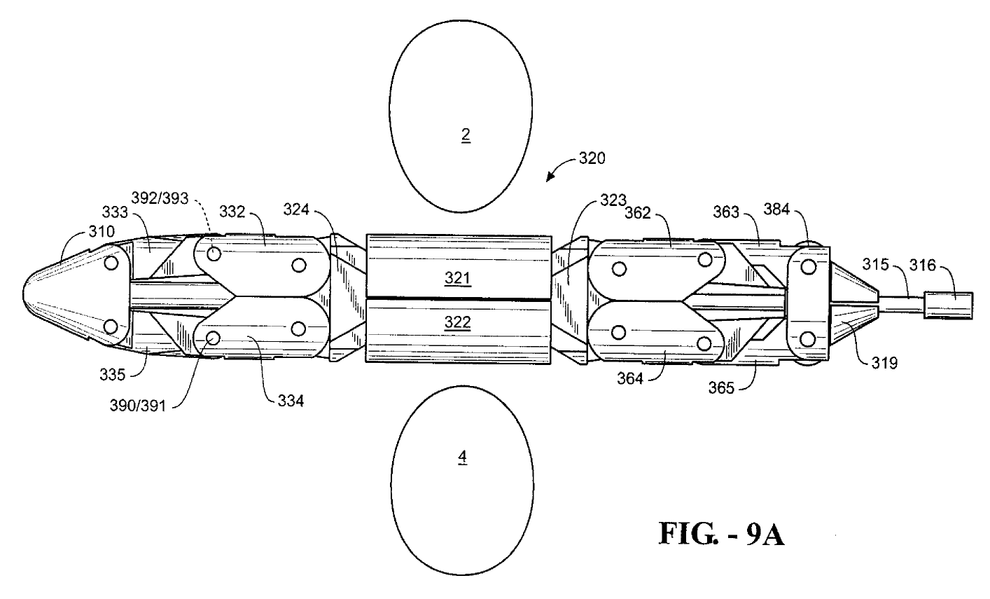

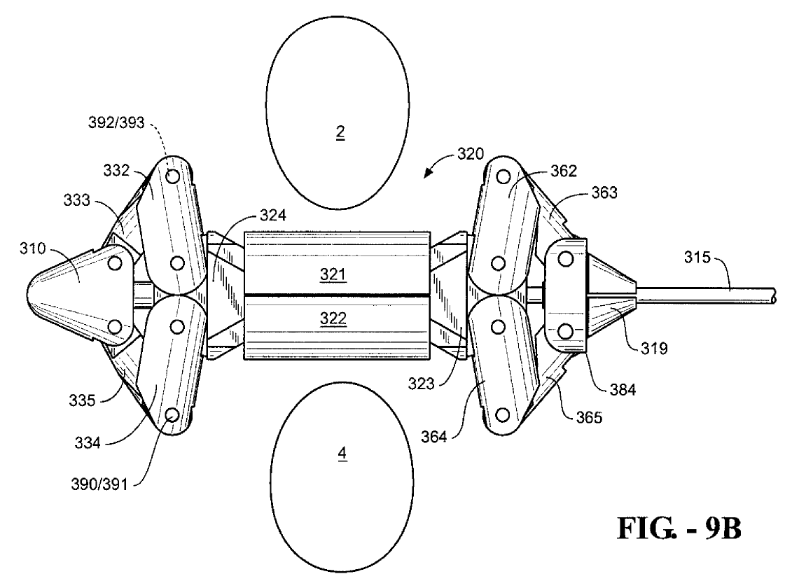

Once the cannula 70 is position, the implant 100 can be urged through the cannula until the distraction guide 110 of the implant 100 is positioned adjacent to the interspinous ligament (Step 106). The implant 100 can then be urged so that the distraction guide 110 forms a space in the interspinous ligament and distracts the fibrous interspinous ligament apart for receipt of the implant 100. The implant 100 is positioned so that the spacer 120 is disposed between the adjacent spinous processes 2,4 (Step 108). Once properly positioned, a rod (also referred to herein as a shaft) 115 connected with the distraction guide 110 and extending through the implant 100 can be urged in a direction opposite a direction of insertion along the longitudinal axis 125 so that the segments 132-135 joining the spacer 120 with the distraction guide 110 pivot away from the rod 115 to form a second wing 130 that resists or limits movement of the implant 100 along the longitudinal axis 125 in a direction opposite a direction of insertion (Step 110). The cannula 70 can be at least partially withdrawn so that segments 162-165 joining the spacer 120 with the end piece 184 are no longer disposed within the cannula 70 (Step 112). With the rod 115 maintained in position, the end piece 184 can be urged in a direction of insertion so that the segments 162-165 connected between the spacer 120 and the end piece 184 pivot away from the rod 115 to form a first wing 160 that resists or limits movement of the implant 100 along the longitudinal axis 125 in the direction of insertion (Step 114). Alternatively, the rod 115 can be urged in a direction opposite a direction of insertion so that the segments 162-165 pivot away from the rod 115 to form a first wing 160 that resists or limits movement of the implant 100 along the longitudinal axis 125 in the direction of insertion. Alternatively, the segments 162-165 can be urged to pivot away from the rod 115 to form a first wing 160 through a combination of urging the rod 115 and urging the end piece 184 in opposite directions. The rod 115 is secured in place by a fastening device 118 (Step 116). For example, in an embodiment the rod 115 can include a bore through which a cotter pin or screw can be positioned to block movement of the rod 115 through the end piece 184. Alternatively a clamp can form a frictional fit with the rod 115. In still further embodiments, the end piece 184 can include a latch and beveled bead, as described below in reference to FIGS. 5 and 8A. In light of these teaching, one of ordinary skill in the art will appreciate the myriad different ways in which the rod 115 can be secured to fix the implant 115 in the second, deployed configuration. Once fixed in position, excess rod 115 can be separated to prevent irritation of associated tissues and structures surrounding the surgical site (Step 118). To ease separation, the rod 115 can optionally include a neck or other weakened portion, for example as described below in reference to FIGS. 5 and 8A. The rod 115 can be snapped off or easily cut at the neck or other weakened portion. The cannula 70 can be withdrawn and the incision closed (Step 120).

In an alternative embodiment, the cannula 70 can be fully removed from over the implant 100 before the first and second wings 160,130 are deployed. In still other embodiments, the cannula can be inserted through the interspinous ligament so that when the implant 100 is positioned at the proximal end of the cannula 70, the cannula 70 need only be retracted over the implant 100 for the implant 100 to be reconfigured to the second, deployed configuration. In light of these teachings, one of ordinary skill in the art will appreciate the myriad different procedural modifications that can be employed to position the implant 100 as desired between adjacent spinous processes 2,4 of the targeted motion segment.

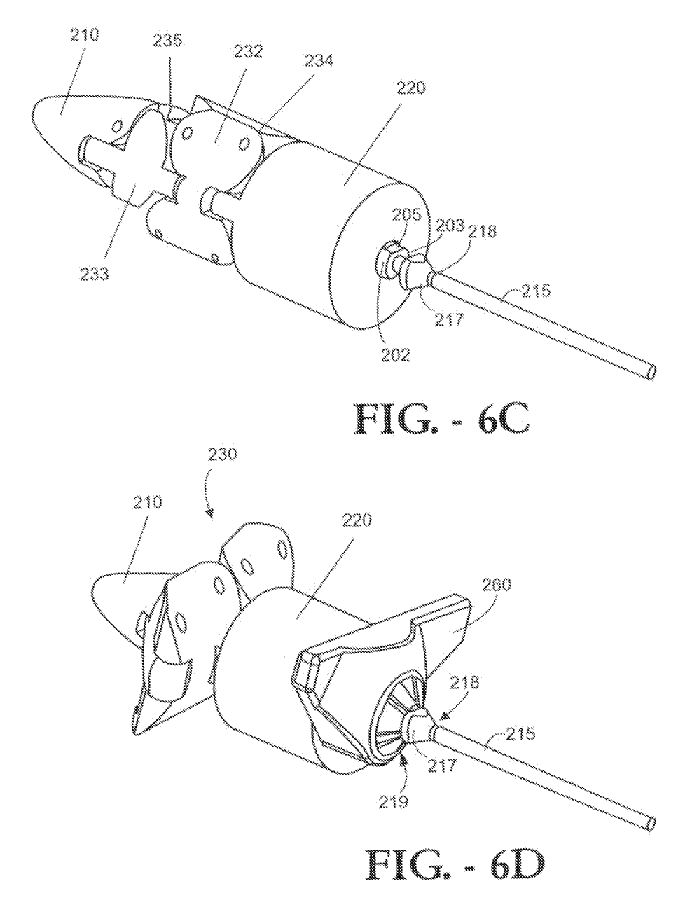

FIG. 5 is a perspective view and FIGS. 6A and 6B are posterior side views of an alternative embodiment of an implant 200 in accordance with the present invention. The implant 200 can comprise a collapsed structure of hinged or otherwise pivotably connected segments 232-235 that pivotably connect a distraction guide 210 and a spacer 220. When deployed (as shown in FIG. 6B), the pivotably connected segments 232-235 form a stop 230 (also referred to herein as a second wing). The second wing 230 resists undesired movement of the implant 200 in a direction opposite a direction of insertion. The implant 200 further includes a fixed first wing 260 from which the spacer 220 extends. As can be seen in FIG. 5, the first wing 260 can have an anterior surface 262 that is beveled to help to avoid tissues. As can be seen, a rod 215 connected with a distraction guide 210 passes through a bore in the spacer 220 and extends through the first wing 260 and a latch 219 extending from the first wing 260. As show, the latch 219 is two or more protruding members biased against the rod 215.

As above, the segments 232-235 include complementary structures 292,293 that can be pivotably connected by pins 290 disposed within holes 291 aligned to receive the pins 290 without obstruction (i.e. they are hinged together). The spacer 220 likewise includes a complementary structure 292 for pivotably joining adjacent segments 232,234. The segments 232-235 are shaped to allow a desired amount of pivoting. For example, the segments 232,234 pivotably connected with the spacer 220 have rounded shapes that together curve generally away from the pins 290 joining the segments 232-235 so that during pivoting, the segments 232-235 have a desired range of motion without obstruction.

The embodiment of FIGS. 5-6B can have a first, collapsed configuration and a second, deployed configuration. Arranged in the first configuration, such implants 200 can include a distraction portion (including the distraction guide 210 and segments 232-235) having a substantially collapsed profile with an approximately uniform thickness. The uniform thickness approximates the thickness of the spacer 220. As shown, in the first, collapsed configuration, the implant 200 has a roughly oval cross-sectional shape approximating a cross-sectional shape of the spacer 220. Referring to FIG. 5, the first, collapsed configuration of the implant 200 allows the implant 200 to be positioned at a surgical site by way of one or more incisions made approaching the interspinous ligament from one side of the interspinous ligament. The distraction guide 210 of the implant 200 can pierce the interspinous ligament and proceed through the interspinous ligament along a longitudinal axis 225, distracting the adjacent spinous processes 2,4 of the targeted motion segment, where desired. The implant 200 can be delivered with the spacer 220 disposed between the adjacent spinous processes 2,4 without the collapsed segments 232-235 substantially obstructing movement along the longitudinal axis 225.

As can be seen more clearly in FIG. 6B, the implant 200 further includes a distal end comprising the latch 219 that can be dilated when passing a feature having a diameter slightly larger than the latch 219. The latch 219 can be used to fix the rod 215 in position once the second wing 230 is deployed. In the embodiment shown, as the rod 215 is urged in a direction opposite a direction of insertion along the longitudinal axis 225, a bead 217 formed along the rod 215 having a diameter wider than the an undilated diameter of the latch 219 can be pulled through the latch 219, causing the latch 219 to briefly expand in diameter until the bead 217 passes through. The latch 219 then closes over the rod 215 to prevent passage of the bead 217 back through the latch 219, thereby fixing the second wing 230 in a deployed position. A bead 217 formed along the rod 215 is a keep and in conjunction with the latch 219 can eliminate a need for a supplemental device for securing the rod, such as a pin, screw, etc. Such a feature can further reduce the complexity of the procedure by eliminating the extra step of securing the rod in place.

Embodiments of implants 200 as shown in FIGS. 5-6B can be at least partially positioned at a surgical site by way of a cannula. As can be seen, the first wing 260 has a shape which is incongruous with that of the spacer 220, and therefore is an obstruction to a cannula having a circumferential shape resembling a cross-sectional shape of the spacer 220. A physician may choose to position the implant 200 in at least two pieces by fixedly associating the first wing 260 with the implant 200 after the spacer 220 is arranged between the adjacent spinous processes. Referring to FIGS. 6C and 6D, the rod 215 of the implant 200 can be threaded through the latch 219 of the first wing 260 until the beveled bead 217 passes through the latch 219, causing the protruding members biased against the rod 215 to block movement of the rod 215 back through the latch 219 in an opposite direction. When the beveled bead 217 is received through the latch, the first wing 260 is fixed in position between the beveled bead 217 and the spacer 220, and limiting movement of the rod 215 relative to the spacer 220. A portion 203 of the rod 215 can include a flat 205 provided for registration of the spacer 220 with the first wing 260.

In an alternative embodiment shown in FIG. 6D, the first wing 260 is fixedly associated with the implant 200 when the beveled bead 217 passes through the latch 219, which can be accomplished be deploying the second wing 230 to thereby shorten a length of the rod 215 that is disposed between the distraction guide 210 and the spacer 220. As above, the segments 232-235 pivot outward to form the second wing 230. Thus, when the beveled bead 217 passes through the latch 219 the implant 200 is configured to resist or limit movement of the spacer 220 relative to the adjacent spinous processes in a direction along the longitudinal axis 225

The first wing 260 can optionally include alignment holes (not shown) on one or more surfaces for allowing an insertion tool to grip the implant 200 (for example as described in U.S. Pat. No. 6,712,819 issued to Zucherman et al).

It should be noted that the embodiment of FIGS. 5-6B need not employ a bead and latch as shown. In other embodiments, an implant having a fixed first wing can further employ a supplemental device for securing the rod. For example, in an alternative embodiment the bore of the spacer can include a spring-loaded ball-bearing that acts as a latch securable to a complementary recess along the rod which acts as a keep. As the rod is drawn or otherwise urged in a direction opposite the direction of implantation, the ball-bearing finds the recess and extends to be captured by the recess. The ball-bearing can resist motion in one or both directions. One of ordinary skill in the art will appreciate upon reflecting on the present teachings that myriad different latch-keep mechanisms can be employed to fix the rod in position relative to the spacer. Implants in accordance with the present inventions are not intended to be limited to a bead and latch as described with particularity above, but are meant to include all structures to secure an actuation device relative to a spacer. Additionally, the embodiment of FIGS. 1-3 need not require a supplemental device for securing the rod, but instead could include the latch extending from the end piece, for example. In such an embodiment, a rod having one or alternatively multiple beads can be employed so that the implant can be deployed in one or more stages. In light of the teachings provided herein, one of ordinary skill in the art can appreciate the myriad different combinations of features which implants falling within the scope of the present invention can employ.

Once the second wing 230 is deployed, the first wing 260 and the second wing 230 restrict or limit movement of the implant 200 along the longitudinal axis 225, preventing the implant 200 from undesirably, and unintentionally being repositioned. The interspinous ligament can help resist anterior-posterior movement of the implant 200 so that the implant 200 remains positioned as desired between the adjacent spinous processes 2,4.

The rod 215 can further include a neck 218 disposed along the rod 215. As shown in FIG. 6B, the neck 218 is arranged between the rod 215 proper and the beveled bead 217 so that the neck 218 is distal of the beveled bead 217. The neck 218 is a portion of the rod 215 that is structurally weaker than the rest of the rod 215 due to its reduced diameter. The rod 215 can more easily be snapped, snipped, or otherwise separated from the beveled bead 217 at the neck 218 once the second wing 230 is deployed and the beveled bead 217 passed through the latch 219. Separating the rod 215 at the neck 218 more cleanly eliminates an excess of rod 215 which may or may not be an irritant to tissues and structures related and adjacent to the targeted motion segment.

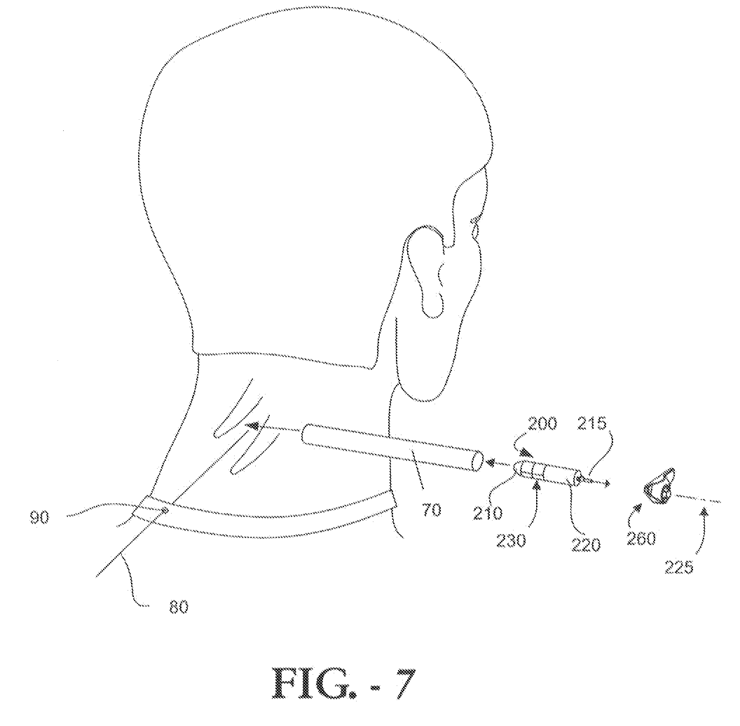

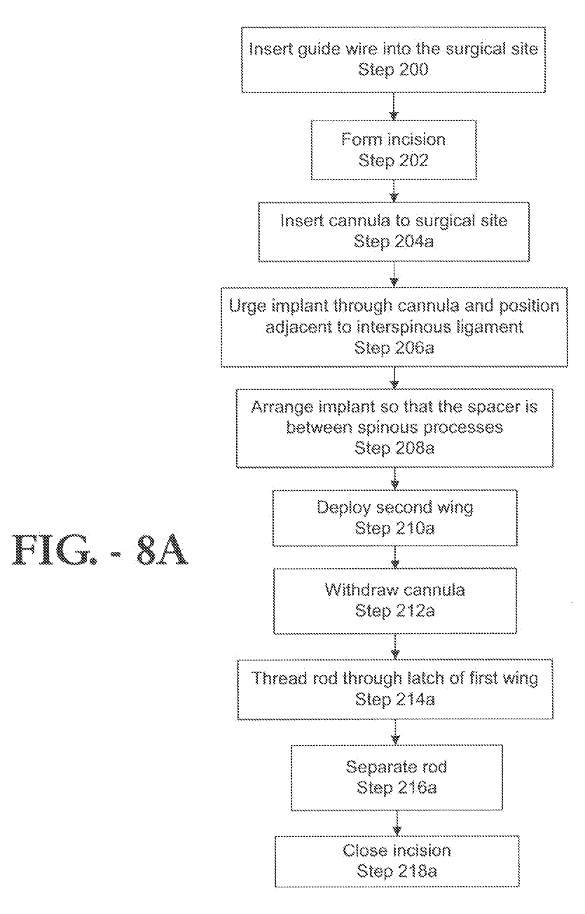

Referring to FIGS. 7 and 8A, in an embodiment of a method of implantation in accordance with the present invention, an incision can be made for accessing a site adjacent to the interspinous ligament of the targeted motion segment. Preferably a guide wire 80 is inserted through a placement network or guide 90 into the surgical site of the implant recipient (e.g. the neck where the targeted motion segment includes cervical vertebra) (Step 200). The guide wire 80 is used to locate where the implant 200 is to be placed relative to the spine, including the spinous processes. Once the guide wire 80 is positioned with the aid of imaging techniques, an incision is made (Step 202) so that the cannula 70 can be positioned through the incision and along a line that is about perpendicular to the guide wire 80 and directed at the end of the guide wire 80 (Step 204a).

Once the cannula 70 is position, the implant 200 can be urged through the cannula 70 until the distraction guide 210 of the implant 200 is positioned adjacent to the interspinous ligament (Step 206a). The implant 200 can then be urged so that the distraction guide 110 forms a space in the interspinous ligament and distracts the fibrous interspinous ligament apart for receipt of the implant 200. The implant 200 is positioned so that the spacer 220 is disposed between the adjacent spinous processes 2,4 (Step 208a). Once properly positioned, a rod 215 connected with the distraction guide 210 and extending through the implant 200 can be urged in a direction opposite a direction of insertion along the longitudinal axis 225 so that the segments 232-235 joining the spacer 220 with the distraction guide 210 pivot away from the rod 215 to form a second wing 230 that resists or limits movement of the implant 200 along the longitudinal axis 225 in a direction opposite a direction of insertion (Step 210a). The cannula 70 can be withdrawn so that the spacer 220 and rod 215 are no longer disposed within the cannula 70 (Step 212a). The first wing 260 can be inserted into the incision, and the rod 215 can be threaded through a latch 219 of the first wing 260 (Step 214a). Once a keep, such as a beveled bead, passes through the latch 219 of the first wing 260, thereby resisting movement of the rod 215 in a direction of implant insertion, the rod 215 can be separated to remove excess material to prevent irritation of associated tissues and structures surrounding the surgical site (Step 216a). To ease separation, the rod 215 can optionally include a neck or other weakened portion, for example as described above. The rod 215 can be snapped off or easily cut at the neck or other weakened portion. The cannula 70 can be withdrawn and the incision closed (Step 218a).

Referring to FIG. 8B, alternatively, once the guide wire 80 is positioned with the aid of imaging techniques, the incision is made (Step 202) so that the implant 200 can be positioned through the incision and along a line that is about perpendicular to the guide wire 80 and directed at the end of the guide wire 80. In such embodiments, the interspinous ligament can optionally be initially distracted using distraction prongs (Step 204b) of a distraction tool, for example such as described in U.S. Pat. Publ. 2006/0036258. The distraction prongs can be held in a distracted position for a prescribed period of time to cause the interspinous ligament to remain at least partially distracted for a generally known period allowing the implant to be positioned within the distraction point of the interspinous ligament. The distraction prongs can optionally provide the further benefit of enabling the space between adjacent spinous processes to be measured, and an appropriately sized implant to be chosen (Step 206b). Once the distraction prongs are removed, the implant 200 (See FIGS. 5-6B) can be positioned adjacent to the spinous ligament, and urged through the interspinous ligament along the longitudinal axis 225 in a first, collapsed configuration (Step 208b). Once the spacer 220 is positioned as desired between the adjacent spinous processes 2,4, the rod 215 can be urged in a direction opposite the direction of insertion along the longitudinal axis 225. As the rod 215 is drawn through the spacer 220 and first wing 260, the segments 231-235 pivot away from the rod 215 to form the second wing 230 (Step 210b)). Once the beveled bead 217 passes through the latch 219 the second wing 230 will be deployed and the rod 215 will be fixed in place. The rod 215 is then snapped or otherwise detached at the neck 218 (Step 212b) and the incision is closed (Step 214b).

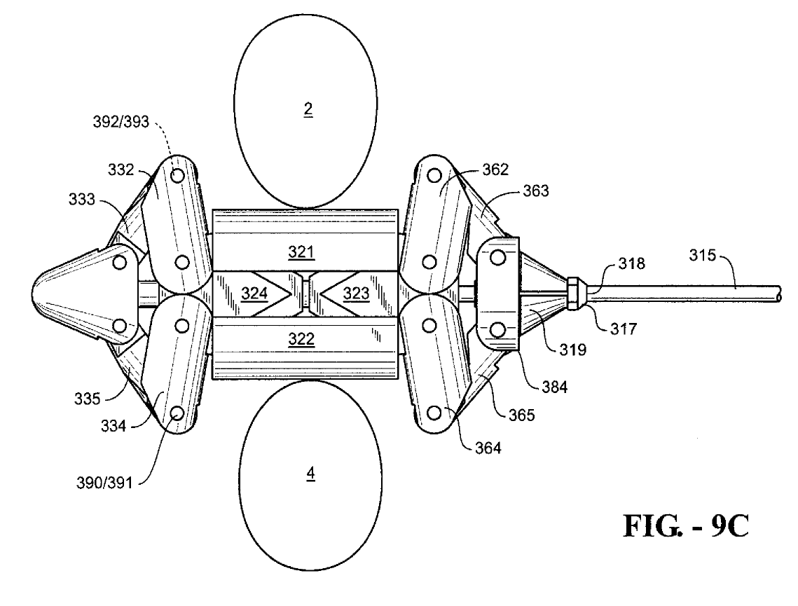

FIGS. 9A-9C are side views of a still further embodiment of an implant 300 in accordance with the present invention. As above, the implant 300 can comprise a collapsed structure of hinged or otherwise pivotably connected segments 332-335,362-365 that when deployed (as shown in FIGS. 9B and 9B) form stops 330,360 (also referred to herein as first and second wings). The first and second wings 360,330 resist undesired movement when the implant 300 is positioned between adjacent spinous processes 2,4. The implant 300 includes a spacer 320 that limits extension motion of two (or more) adjacent spinous processes 2,4 by resisting compressive forces applied to the spacer 320 by the adjacent spinous processes 2,4. The spacer 320 limits movement to preferably limit the collapse of the foraminal canal within which nerves are disposed. The spacer 320 comprises an upper seat 321, a lower seat 322, a first distraction piece 323 and a second distraction piece 324.

As above, the segments 332-335,362-365 include complementary structures 392,393 that can be pivotably connected by pins 390 disposed within holes 391 aligned to receive the pins 390 without obstruction (i.e. they are hinged together). The first distraction piece 323 and second distraction piece 324 likewise includes a complementary structure for pivotably joining adjacent segments 332,334,362,364. Still further, an end piece 384 and a distraction guide 310 include complementary structures for pivotably joining adjacent segments 333,335, 363,365. As can be seen, a rod 315 connected with the distraction guide 330 passes through a bore in the spacer 320 and passes through a latch 319 extending from the end piece 384. The rod 315 as shown includes a knob 316 for gripping the rod 315 to ease manipulation of the rod 315. In other embodiments a knob 316 need not be employed. As show, the latch 319 is two or more segmented members biased against the rod 315. The segments 332-335,362-365 are shaped to allow a desired amount of pivoting. For example, the segments 332,334,362,364 pivotably connected with the spacer 320 have rounded shapes that together that curve substantially away from the pins 390 joining the segments 332-335,362-365 so that during pivoting, the segments 332-335,362-365 have a desired range of motion without obstruction.

The embodiment of FIGS. 9A-9C can have a first, collapsed configuration, a second, partially deployed configuration (as shown in FIG. 9B), and a third, configuration wherein a height of the spacer 320 is expanded. Arranged in the first configuration, such implants 300 can have a substantially collapsed profile having an approximately uniform thickness. The uniform thickness approximates the thickness of the spacer 320 having an unexpanded height. Referring to FIG. 3, the first, collapsed configuration of the implant 300 allows the implant 300 to be positioned at a surgical site by way of one or more incisions made approaching the interspinous ligament from one side of the interspinous ligament. The distraction guide 310 of the implant 300 can pierce the interspinous ligament and proceed through the interspinous ligament along a longitudinal axis 325, distracting the adjacent spinous processes 2,4 of the targeted motion segment, where desired.

The spacer 320 has a height that can be expanded after the implant 300 has been positioned between the targeted adjacent spinous processes. In an embodiment, the spacer 320 can be expanded to a height to achieve a desired minimum distance between adjacent spinous processes during extension motion (referred to hereinafter as a target height). In an undeployed configuration (see FIG. 9A), the spacer 320 can have a height smaller than the target height, thereby reducing the cross-sectional area of the spacer 320 disposed about an axis of insertion. A smaller cross-sectional area of the spacer 320 can reduce an amount of trauma affecting the adjacent spinous processes and related tissue and structures. The smaller cross-sectional area can further ease positioning of the implant 300 by reducing the amount force required to be applied in displacing tissue and other structures to accommodate the implant 300. Where a cannula is employed, a diameter and/or cross-sectional shape of the cannula can be reduced to a size that is roughly the maximum cross-sectional area of the undeployed implant. The implant 300 can be delivered with the spacer 320 disposed between the adjacent spinous processes 2,4 without the collapsed segments 362-365 substantially obstructing movement along the longitudinal axis 325. It can be preferable to employ a cannula having a smaller cross-section area to reduce trauma to structures and tissues during insertion.

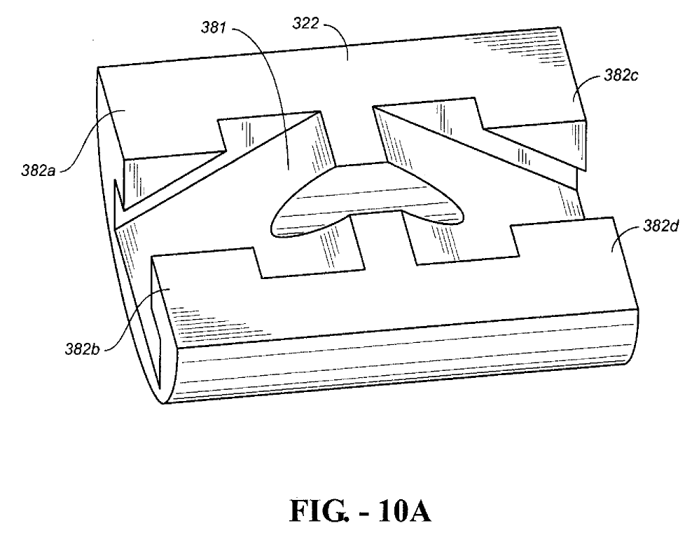

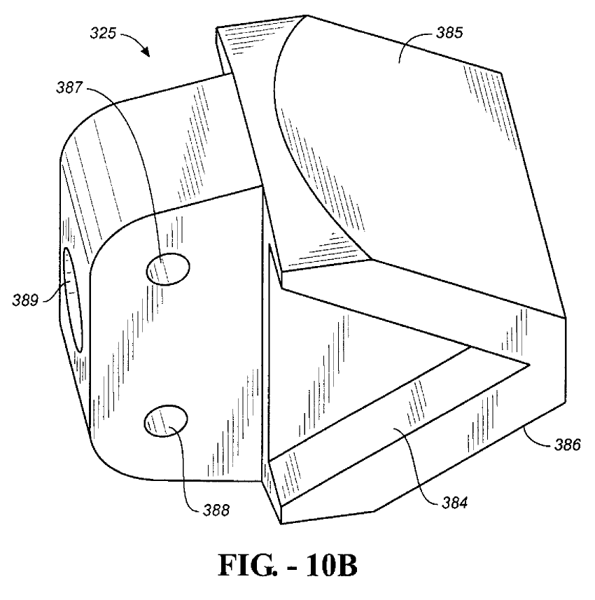

The height of the spacer can be expanded during actuation of the rod. Height expansion can be achieved by translating a portion of the motion along the longitudinal axis to a component of motion perpendicular to the longitudinal axis. In an embodiment, motion can be translated using ramped surfaces. Referring to FIGS. 10A and 10B, a lower seat 322 of the spacer can include an inner structure 380 that includes a ramp 381. The first distraction piece 323 moves along the ramp 381 of the inner structure 380 and includes a flange 384 that is captured by retaining structures 382a, 382b of the lower seat 322. The first distraction piece 323 as shown has an upper ramped surface 385 and a lower ramped surface 386. In other embodiments, the first distraction piece 323 can have one of the upper and lower ramped surface and a flat surface. In such embodiments, an amount of extension is reduced. Likewise, the first distraction piece 323 and inner structure 380 can have complementary shapes other than as shown in FIGS. 10A and 10B, for example the first distraction piece 323 and inner structure 380 can have ramped shapes having a larger or smaller angle relative to the longitudinal axis. As shown, the first distraction piece 323 includes two bores 387,388 for receiving pins (not shown) for pivotably connecting segments. Further, a bore 389 is provided through the first distraction piece 323 for receiving a rod. While the upper seat is not illustrated, the upper seat will have a shape and structure that accommodates the first distraction piece 323 and second distraction piece in a similar manner as has been described with the lower seat 322. That is, the upper seat can be shaped to enable a desired expansion of overall spacer height. It will be appreciated by one of ordinary skill in the art in light of these teachings, that the structures of the spacer need not appear as shown in FIGS. 9A-10B, but rather can be any structures that actuatable by motion of a rod to expand in height to a target height.

Referring to FIGS. 10B and 10C, expansion of the spacer 320 height can be achieved by urging the rod 315 in a direction along the longitudinal axis 325 in a direction opposite a direction of implantation, urging the distraction guide 310 toward the latch 319. As the length of rod 315 disposed within the implant 300 shortens, the first distraction piece 323 and the second distraction piece 324 are urged toward each other, sliding up the ramped surface 385 so that the upper seat 321 and lower seat 322 are wedged apart, thereby expanding the height of the implant 300. As shown, the rod 315 can include a beveled bead or other keep that can be retained in position by a latch 319. Although not shown, the rod 315 can include multiple beads for fixing the rod 315 in position for a plurality of heights of the spacer 320. Thus, a target height may not be known with exactness by the physician at the time of implantation, but rather is assessed during deployment. Further, employing multiple beads can assist a physician by preventing collapse of the entire structure where the rod is released or otherwise no longer actuated. As above, a necked structure can be arranged at the one or more beads to allow the rod 315 to be trimmed.

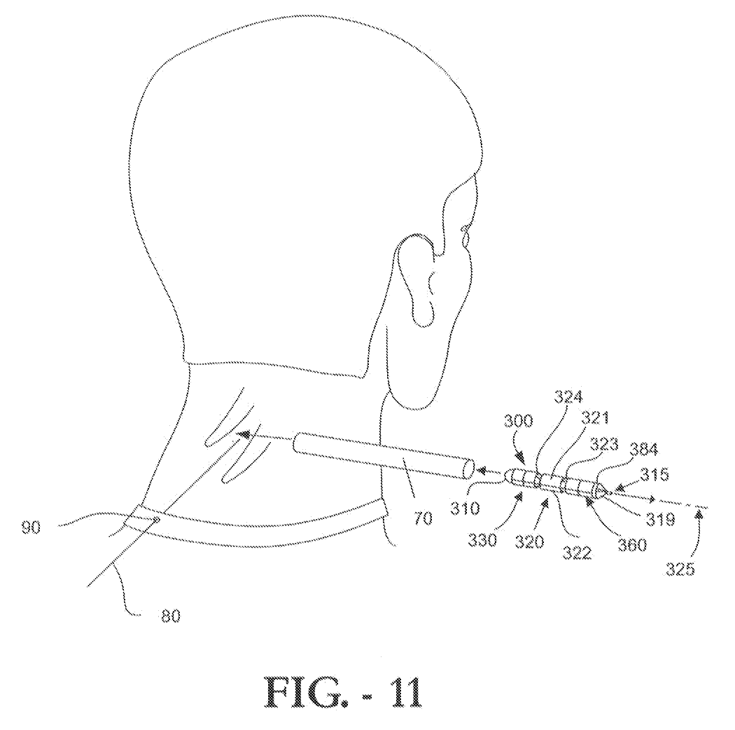

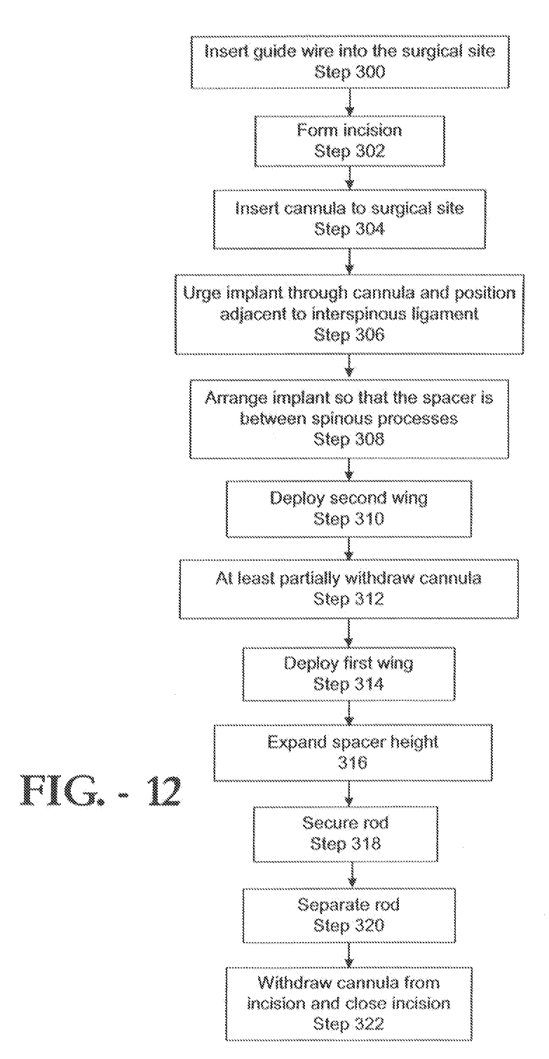

As above, referring to FIGS. 11 and 12 in an embodiment of a method of implantation in accordance with the present invention, the cannula can be positioned adjacent to the interspinous ligament of the targeted motion segment. Preferably a guide wire 80 is inserted through a placement network or guide 90 into the surgical site of the implant recipient (e.g. the neck where the targeted motion segment includes cervical vertebra) (Step 300). The guide wire 80 is used to locate where the implant 300 is to be placed relative to the spine, including the spinous processes. Once the guide wire 80 is positioned with the aid of imaging techniques, an incision is made (Step 302) so that the cannula can be positioned through the incision and along a line that is about perpendicular to the guide wire 80 and directed at the end of the guide wire 80 (Step 304).

Once the cannula 70 is positioned, the implant 300 can be urged through the cannula 70 until the distraction guide 310 of the implant 300 is positioned adjacent to the interspinous ligament (Step 306). The implant 300 can then be urged so that the distraction guide 310 forms a space in the interspinous ligament and distracts the fibrous interspinous ligament apart for receipt of the implant 300. The implant 300 is positioned so that the spacer 320 is disposed between the adjacent spinous processes 2,4 (Step 308). Once properly positioned, a rod 315 connected with the distraction guide 310 and extending through the implant 300 can be urged in a direction opposite a direction of insertion along the longitudinal axis 325 so that the segments joining a second distraction piece 324 of the spacer 320 with the distraction guide 310 pivot away from the rod 315 to form a second wing 330 that resists or limits movement of the implant 300 along the longitudinal axis 325 in a direction opposite a direction of insertion (Step 310). The cannula 70 can be at least partially withdrawn so that the upper seat 321 and lower seat 322 are no longer disposed within the cannula 70 (Step 312). The rod 315 can then be further urged in a direction opposite a direction of insertion so that the upper seat 321 and lower seat 322 are urged apart, expanding the height of the spacer 320 to a target height (step 314). The cannula 70 can further withdrawn so that segments joining a first distraction piece 323 of the spacer 320 with the end piece 384 are no longer disposed within the cannula 70 (Step 316). The rod 315 can then be still further urged in a direction opposite a direction of insertion so that the segments pivot away from the rod 315 to form a first wing 360 that resists or limits movement of the implant 300 along the longitudinal axis 325 in the direction of insertion (Step 318). The rod 315 is secured in place when a bead (not shown) formed along the rod 315 is urged through a latch 319, which then closes over the bead to resist movement of the rod 315 in the direction of insertion (Step 318). Once fixed in position, excess rod 115 can be separated to prevent irritation of associated tissues and structures surrounding the surgical site (Step 320). The cannula can be withdrawn and the incision closed (Step 322).

In an alternative embodiment, the cannula 70 can be fully removed from over the implant 300 before the first and second wings 360,330 and the spacer seats 321,322 are deployed.

### Materials for Use in Implants of the Present Invention

In some embodiments, the implant can be fabricated from medical grade metals such as titanium, stainless steel, cobalt chrome, and alloys thereof, or other suitable implant material having similar high strength and biocompatible properties. Additionally, the implant can be at least partially fabricated from a shape memory metal, for example Nitinol, which is a combination of titanium and nickel. Such materials are typically radiopaque, and appear during x-ray imaging, and other types of imaging. Implants in accordance with the present invention, and/or portions thereof can also be fabricated from somewhat flexible and/or deflectable material. In these embodiments, the implant and/or portions thereof can be fabricated in whole or in part from medical grade biocompatible polymers, copolymers, blends, and composites of polymers. A copolymer is a polymer derived from more than one species of monomer. A polymer composite is a heterogeneous combination of two or more materials, wherein the constituents are not miscible, and therefore exhibit an interface between one another. A polymer blend is a macroscopically homogeneous mixture of two or more different species of polymer. Many polymers, copolymers, blends, and composites of polymers are radiolucent and do not appear during x-ray or other types of imaging. Implants comprising such materials can provide a physician with a less obstructed view of the spine under imaging, than with an implant comprising radiopaque materials entirely. However, the implant need not comprise any radiolucent materials.

One group of biocompatible polymers are the polyaryl ester ketones which has several members including polyetheretherketone (PEEK), and polyetherketoneketone (PEKK). PEEK is proven as a durable material for implants, and meets the criterion of biocompatibility. Medical grade PEEK is available from Victrex Corporation of Lancashire, Great Britain under the product name PEEK-OPTIMA. Medical grade PEKK is available from Oxford Performance Materials under the name OXPEKK, and also from CoorsTek under the name BioPEKK. These medical grade materials are also available as reinforced polymer resins, such reinforced resins displaying even greater material strength. In an embodiment, the implant can be fabricated from PEEK 450G, which is an unfilled PEEK approved for medical implantation available from Victrex. Other sources of this material include Gharda located in Panoli, India. PEEK 450G has the following approximate properties:

| Property | Value |
|---|---|
| Density | 1.3 g/cc |
| Rockwell M | 99 |
| Rockwell R | 126 |
| Tensile Strength | 97 MPa |
| Modulus of Elasticity | 3.5 GPa |
| Flexural Modulus | 4.1 GPa |

PEEK 450G has appropriate physical and mechanical properties and is suitable for carrying and spreading a physical load between the adjacent spinous processes. The implant and/or portions thereof can be formed by extrusion, injection, compression molding and/or machining techniques.

It should be noted that the material selected can also be filled. Fillers can be added to a polymer, copolymer, polymer blend, or polymer composite to reinforce a polymeric material. Fillers are added to modify properties such as mechanical, optical, and thermal properties. For example, carbon fibers can be added to reinforce polymers mechanically to enhance strength for certain uses, such as for load bearing devices. In some embodiments, other grades of PEEK are available and contemplated for use in implants in accordance with the present invention, such as 30% glass-filled or 30% carbon-filled grades, provided such materials are cleared for use in implantable devices by the FDA, or other regulatory body. Glass-filled PEEK reduces the expansion rate and increases the flexural modulus of PEEK relative to unfilled PEEK. The resulting product is known to be ideal for improved strength, stiffness, or stability. Carbon-filled PEEK is known to have enhanced compressive strength and stiffness, and a lower expansion rate relative to unfilled PEEK. Carbon-filled PEEK also offers wear resistance and load carrying capability.

As will be appreciated, other suitable similarly biocompatible thermoplastic or thermoplastic polycondensate materials that resist fatigue, have good memory, are flexible, and/or deflectable, have very low moisture absorption, and good wear and/or abrasion resistance, can be used without departing from the scope of the invention. As mentioned, the implant can be comprised of polyetherketoneketone (PEKK). Other material that can be used include polyetherketone (PEK), polyetherketoneetherketoneketone (PEKEKK), polyetheretherketoneketone (PEEKK), and generally a polyaryletheretherketone. Further, other polyketones can be used as well as other thermoplastics. Reference to appropriate polymers that can be used in the implant can be made to the following documents, all of which are incorporated herein by reference. These documents include: PCT Publication WO 02/02158 A1, dated Jan. 10, 2002, entitled “Bio-Compatible Polymeric Materials;” PCT Publication WO 02/00275 A1, dated Jan. 3, 2002, entitled “Bio-Compatible Polymeric Materials;” and, PCT Publication WO 02/00270 A1, dated Jan. 3, 2002, entitled “Bio-Compatible Polymeric Materials.” Other materials such as Bionate®, polycarbonate urethane, available from the Polymer Technology Group, Berkeley, Calif., may also be appropriate because of the good oxidative stability, biocompatibility, mechanical strength and abrasion resistance. Other thermoplastic materials and other high molecular weight polymers can be used.

The foregoing description of the present invention have been presented for purposes of illustration and description. It is not intended to be exhaustive or to limit the invention to the precise forms disclosed. Many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

What is claimed:

1. An interspinous implant adapted to be inserted between spinous processes, the implant comprising:
a first wing;
a spacer extending from the first wing, the spacer having a thickness; and
a second wing extending from the spacer, the second wing having a first configuration and the second wing selectably arrangeable in a second configuration;
wherein in the first configuration the second wing has a height that is approximately the same as the thickness of the spacer;
wherein in the second configuration the second wing has a height that is greater than the thickness of the spacer.

2. The implant of claim 1, further comprising:
a hole extending through the spacer and the first wing;
a rod disposed within the hole, the rod being used to selectably arrange the second wing to the first configuration and the second configuration when moved relative to the spacer.

3. The implant of claim 2, further comprising:
a distraction guide;
wherein the second wing is pivotably connected between the distraction guide and the spacer.

4. The implant of claim 1, wherein:
the second wing is pivotably connected between the distraction guide and the spacer by a plurality of pins;
the second wing includes a plurality of holes;
the distraction guide includes a hole aligned with a corresponding hole of the plurality of holes;
the spacer includes a hole aligned with a corresponding hole of the plurality of holes; and
the plurality of pins are disposed within the plurality of holes.

5. The implant of claim 2, further comprising:
a latch having an opening;
a bead disposed along a length of the rod;
wherein the rod extends through the opening;
wherein the bead has a diameter larger than the opening.

6. The implant of claim 5, wherein when the bead is urged through the latch in a direction opposite a direction of insertion, the second wing is secured in the second configuration.

7. The implant of claim 5 further comprising:
a neck disposed along a length of the rod;
wherein the rod is severable at the neck.

8. The implant of claim 1, wherein:
the spacer includes an upper seat and a lower seat;
the thickness is a first thickness; and
the upper seat and the lower seat can be urged apart so that the spacer has a second thickness.

9. The implant of claim 1, wherein:
the first wing has a first configuration and the first wing is selectably arrangeable in a second configuration;
in the first configuration the first wing has a height that is approximately the same as the thickness of the spacer; and
in the second configuration the first wing has a height that is greater than the thickness of the spacer.

10. A system for supporting adjacent spinous processes, the system comprising:
an implant including:
a first wing having a first configuration and the first wing arrangeable in a second configuration;
wherein in the first configuration the first wing has a height that is approximately the same as the thickness of the spacer; and
wherein in the second configuration the first wing has a height that is greater than the thickness of the spacer.
a spacer extending from the first wing, the spacer having a thickness; and
a second wing extending from the spacer, the second wing having a first configuration and the second wing selectably arrangeable in a second configuration;
wherein in the first configuration the second wing has a height that is approximately the same as the thickness of the spacer;
wherein in the second configuration the second wing has a height that is greater than the thickness of the spacer.
a distraction tool including distraction prongs having a proximate end positionable between the adjacent spinous processes and adapted to pierce and distract an interspinous ligament disposed between the adjacent spinous processes.

11. The implant of claim 10, further comprising:
a hole extending through the spacer and the first wing;
a rod disposed within the hole, the rod being used to selectably arrange the second wing to the first configuration and the second configuration when moved relative to the spacer.

12. The implant of claim 10, further comprising:
a distraction guide;
wherein the second wing is pivotably connected between the distraction guide and the spacer.

13. The implant of claim 10, wherein:
the spacer includes an upper seat and a lower seat;
the thickness is a first thickness; and
the upper seat and the lower seat can be urged apart so that the spacer has a second thickness.

14. The implant of claim 10, wherein:
the second wing is pivotably connected between the distraction guide and the spacer by a plurality of pins;
the second wing includes a plurality of holes;
the distraction guide includes a hole aligned with a corresponding hole of the plurality of holes;
the spacer includes a hole aligned with a corresponding hole of the plurality of holes; and
the plurality of pins are disposed within the plurality of holes.

15. The implant of claim 10, further comprising:
a latch having an opening;
a bead disposed along a length of the rod;
wherein the rod extends through the opening;
wherein the bead has a diameter larger than the opening.

16. The implant of claim 15, wherein when the bead is urged through the latch in a direction opposite a direction of insertion, the second wing is secured in the second configuration.

17. The implant of claim 15, further comprising:
a neck disposed along a length of the rod;
wherein the rod is severable at the neck.

18. The implant of claim 10, wherein:
the distraction tool is adapted to measure a length of a space between the adjacent spinous processes;
the thickness of the spacer is selectable based on the length.

19. A method of arranging an implant between adjacent spinous processes, the method comprising:
forming an incision at a surgical site such that an interspinous ligament disposed between the adjacent spinous processes is accessible from one side of the interspinous ligament;
piercing the interspinous ligament with a distraction tool;
positioning an implant between the adjacent spinous processes;
urging the implant through into the distracted space, the implant including a first wing, a spacer having a thickness, and a second wing, wherein a first configuration of the second wing has a first height substantially similar to the thickness;
arranging the spacer between the adjacent spinous processes;
rearranging the second wing to a second configuration such that the wing has a second height greater than the first height;
closing the incision.

20. The method of claim 19, further comprising:
measuring a length of a space between the adjacent spinous processes;
selecting an implant having a thickness based on the length.

21. The method of claim 19, wherein rearranging the wing to a second configuration includes urging a rod operably associated with the implant in a direction opposite the direction of urging while generally maintaining the implant in position.

22. An interspinous implant adapted to be inserted between spinous processes, the implant comprising:
a spacer including an upper seat and a lower seat having an initial height at a first spacer configuration, the spacer selectably arrangeable in a second spacer configuration having a height greater than the initial height; and
a wing connected with the spacer, the wing having a first configuration and the wing selectably arrangeable in a second configuration;
wherein in the first configuration the wing has a height that is approximately the same as the initial height of the spacer;
wherein in the second configuration the wing has a height that is greater than the height of the spacer in the second configuration.

23. An interspinous implant adapted to be inserted between spinous processes, the implant comprising:
a first wing having a first configuration and the first wing arrangeable in a second configuration;
a spacer extending from the first wing, the spacer including an upper seat and a lower seat having an initial height at a first spacer configuration, the spacer selectably arrangeable in a second spacer configuration having a height greater than the initial height;
wherein in the first configuration the first wing has a height that is approximately the same as the thickness of the spacer; and
wherein in the second configuration the first wing has a height that is greater than the thickness of the spacer; and
a second wing connected with the spacer, the wing having a first configuration and the wing selectably arrangeable in a second configuration;
wherein in the first configuration the second wing has a height that is approximately the same as the initial height of the spacer; and
wherein in the second configuration the second wing has a height that is greater than the height of the spacer in the second configuration.

24. An interspinous implant adapted to be inserted between spinous processes, the implant comprising:
a first wing;
a spacer extending from the first wing, the spacer including an upper seat and a lower seat having an initial height at a first spacer configuration, the spacer selectably arrangeable in a second spacer configuration having a height greater than the initial height; and
a second wing.
